(19) United States
(12) Patent Application Publication
(10) Pub. No.: US 2004/0121010 A1
(43) Pub. Date: Jun. 24, 2004

(54) Pulsatile release compositions of milnacipran

(75) Inventors: Jane Hirsh, Wellesley, MA (US); Roman V. Rariy, Allston, MA (US); Michael Heffernan, Hingham, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Collegium Pharmaceutical, Inc.

(21) Appl. No.: 10/690,872

(22) Filed: Oct. 22, 2003

Related U.S. Application Data
(60) Provisional application No. 60/421,640, filed on Oct. 25, 2002.
(60) Provisional application No. 60/431,626, filed on Dec. 5, 2002.
(60) Provisional application No. 60/431,627, filed on Dec. 5, 2002.
(60) Provisional application No. 60/431,906, filed on Dec. 9, 2002.
(60) Provisional application No. 60/431,861, filed on Dec. 9, 2002.
(60) Provisional application No. 60/443,618, filed on Jan. 29, 2003.
(60) Provisional application No. 60/459,061, filed on Mar. 28, 2003.
(60) Provisional application No. 60/458,994, filed on Mar. 28, 2003.
(60) Provisional application No. 60/458,995, filed on Mar. 28, 2003.

(51) Int. Cl. A61K 9/22; A61K 31/165

(52) U.S. Cl. 424/468; 514/620

(57) ABSTRACT

A once-a-day oral milnacipran pulsatile release composition has been developed that releases the drug in spaced apart “pulses”. The dosage forms are comprised of first, second and optional third dosage units, with each dosage unit having a different drug release profile. This dosage form provides in vivo drug plasma levels characterized by Cₘₐₓ below 3000 ng/ml, preferably below 2000 ng/ml, and most preferably below 1000 ng/ml. These levels help to avoid stimulation of the cholinergic effects on the CNS. The composition allows milnacipran to be delivered over approximately 24 hours, when administered to a patient in need, resulting in diminished incidence or decreased intensity of common milnacipran side effects such as sleep disturbance, nausea, vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

This application claims priority under 35 U.S.C. 119 to U.S. Ser. No. 60/421,640 filed Oct. 25, 2002; U.S. Ser. No. 60/431,626 filed Dec. 5, 2002; U.S. Ser. No. 60/431,627 filed Dec. 5, 2002; U.S. Ser. No. 60/431,906 filed Dec. 9, 2002; U.S. Ser. No. 60/431,861 filed Dec. 9, 2002; U.S. Ser. No. 60/443,618 filed Jan. 29, 2003; U.S. Ser. No. 60/459,061 filed Mar. 28, 2003; U.S. Ser. No. 60/458,994 filed Mar. 28, 2003; U.S. Ser. No. 60/458,995 filed Mar. 28, 2003.

### FIELD OF THE INVENTION

The present invention generally relates to novel milnacipran pulsatile release compositions.

### BACKGROUND OF THE INVENTION

Efficacy and tolerability are important factors determining the choice of a medication for treatment of mental depression and other mental disorders including Functional Somatic Disorders. The move from tricyclic antidepressants (TCAs) to selective serotonin reuptake inhibitors (SSRIs) involved not only the loss of the direct receptor interactions responsible for the adverse side effects of TCAs, but also the ability to inhibit the reuptake of norepinephrine. Selectivity for the single neurotransmitter, serotonin, may explain why SSRIs tend to be less efficacious than the TCAs, especially in more serious forms of depression (Lopez-Ibor J. et al., 1996, Int. Clin. Psychopharm., 11:41-46). Older TCAs are associated with significant behavioral toxicity, notably psychomotor and cognitive impairment and sedation. SSRIs are largely devoid of these effects, but gastrointestinal disturbances such as nausea and dyspepsia are common with these agents (Hindmarch I., 1997, Human Psychopharmacology, 12:115-119). For example, for widely prescribed SSRI sertraline (Zoloft®, Pfizer) the top three adverse events associated with discontinuation of treatment were nausea, insomnia, and diarrhea (Physician's Desk Reference, 57^{th} Edition, 2003, Thomson Medical).

Efforts toward improving antidepressant medications are guided by cumulative evidence from neurochemical and clinical studies supporting the therapeutic potential of enhancing monoamine function in depression. A number of antidepressant drugs, serotonin and norepinephrine reuptake inhibitors (SNRIs), including duloxetine, venlafaxine, and milnacipran, have been developed based on their interaction with both serotonin (5-HT) and norepinephrine (NE) receptors. Milnacipran is more appropriately referred to as norepinephrine and serotonin reuptake inhibitor (NSRI) since its norepinephrine (“NE”) to serotonin (“5-HT”) ratio is 2:1 (Moret et al., 1985, Neuropharmacology, 24:1211-1219; Palmier et al., 1989, Eur. J. Clin. Pharmacol., 37:235-238). Current clinical evidence suggests that these new agents may offer improved efficacy and/or faster onset of action compared with SSRIs (Tran P. V. et al., 2003, J. Clin. Psychopharmacol., 23:78-86). Recent trials with NSRI milnacipran suggest that this compound is effective in relieving pain both associated with, and independent of, depression (Briley M., 2003, Curr. Opin. Investig. Drugs, 4:42-45; Cypress Bioscience Inc., Cypress Bioscience Inc. Announces Final Results of Milnacipran Phase II Clinical Trial in Fibromyalgia, Media Release, Mar. 21, 2003, Available from: URL: http://www.cypressbio.com).

Unfortunately these SNRI and NSRI compounds have demonstrated numerous side effects in human clinical trials.

For example, the safety and tolerability of duloxetine (Cymbalta®, Eli Lilly and Company) was assessed in a pooled analysis of 7 double-blind trials involving 1032 patients treated with duloxetine (40-120 mg/day) and 732 patients treated with placebo. Adverse events which occurred at a rate of more than 5% for duloxetine were nausea, dry mouth, fatigue, dizziness, constipation, somnolence, decreased appetite, and sweating. Adverse events which led to discontinuation of treatment were nausea, dizziness, somnolence, dermatitis, insomnia, headache, and fatigue. Nausea and dizziness led to significantly more duloxetine-treated patients discontinuing treatment, compared with placebo (Mallinckrodt C. et al., American Psychiatric Association 2002 Annual Meeting, New Research Abstracts, 119, May 18, 2002; Detke M. J. et al., American Psychiatric Association 2002 Annual Meeting, New Research Abstracts, 33-34, May 18, 2002). Nausea was the only adverse event reported as a reason for discontinuation (Eli Lilly and Company, New Research Shows Cymbalta Reduces Anxiety Symptoms Associated With Depression, Media Release: Sep. 18, 2003, Available from: URL:.

For venlafaxine (Effexor®, Wyeth-Ayerst), a member of the SNRI family, major reported side effects are the ones that affected the gastrointestinal system. In 4- to 8-week placebo-controlled clinical trials treatment-emergent major gastrointestinal adverse experience incidence for Effexor® versus placebo (n=1,033 vs. 609) were: nausea (37% vs. 11%), constipation (15% vs. 7%), anorexia (11% vs. 2%), and vomiting (6% vs. 2%). In the same clinical trials treatment-emergent major central nervous system adverse experience incidence were: somnolence (23% vs. 9%), dry mouth (22% vs. 11%), dizziness (19% vs 7%), insomnia (18% vs. 10%), nervousness (13% vs. 6%), anxiety (6% vs. 3%), tremor (5% vs. 1%). Importantly, nausea, in addition to being the most common reported side effect (see above), was the top reason venlafaxine patients in Phase 2 and Phase 3 depression studies discontinued treatment: almost 32% of patients who discontinued treatment did so due to nausea (Physician's Desk Reference, 57th Edition, 2003, Thomson Medical).

Milnacipran (Ixel®, Pierre Fabre), has demonstrated numerous adverse reactions in human clinical trials with tolerability decreasing with increasing dose (Puech A. et al., 1997, Int. Clin. Psychopharm., 12:99-108). In the double-blind, randomized, multicenter clinical study the most frequent spontaneously reported adverse events for 100 mg/day milnacipran twice daily were as follows: abdominal pain (13%), constipation (10%), and headache (9%). Interestingly, when in the same study milnacipran was given 200 mg/day twice daily, pain related adverse reactions decreased (headache to 8% and abdominal pain to 7%) but nausea and vomiting were more pronounced side effects and were reported by 7% of the patients (Guelfi J. D., 1998, Int. Clin. Psychopharm., 13:121-128). In a double-blind comparative study involving 219 elderly patients with depression the only adverse event reported more frequently for milnacipran recipients than for TCA imipramine recipients was nausea. Patients received either milnacipran or imipramine 75-100 mg/day twice daily for 8 weeks (Tignol J. et al., 1998, Acta Psychiatrica Scandinavica, 97:157-165). It was also observed that when milnacipran was administered intravenously to 10 patients, five of them reported transient nausea. Nausea was primarily reported at the moment of peak of milnacipran plasma level (Caron J. et al., 1993, Eur. Neuropsychopharmacol., 3:493-500). This study clearly demonstrates that nausea is directly correlated with the milnacipran blood plasma concentration. In addition, it strongly suggests that the nausea can be a centrally mediated side effect since the drug was given intravenously in this study. Data from other studies suggest that milnacipran may also induce a locally mediated nausea via gastric irritation (the rapid onset of the nausea was observed even prior to achieving peak plasma levels).

The incidence of spontaneously reported milnacipran adverse experiences in placebo-controlled clinical trials is given in Table 1 (adverse effect is listed if frequency was more than 2% in milnacipran 100 mg/day group). As it can be clearly seen from data presented in Table 1, the incidence of certain adverse events increases with dosage, including nausea, vomiting, sweating, hot flashes, palpitations, tremor, anxiety, dysuria, and insomnia.

TABLE 1
Incidence of spontaneously reported milnacipran adverse
experiences in placebo-controlled clinical trials

| | Frequency of Adverse Experiences (%) | | | |
|---|---|---|---|---|
| | | 50 mg/day | 100 mg/day | 200 mg/day |
| Adverse | Placebo | twice daily | twice daily | twice daily |
| Event | N = 394 | N = 426 | N = 1871 | N = 865 |
| Nausea | 10.9 | 12.7 | 11.2 | 19.4* |
| Headache | 17.0 | 14.6 | 8.4 | 13.5 |
| Increased | 1.3 | 14.0 | 4.3* | 11.6* |
| Sweating | | | | |
| Constipation | 4.3 | 8.0 | 6.5 | 11.4* |
| Insomnia | 10.7 | 9.2 | 6.1 | 11.3 |
| Dry mouth | 5.6 | 9.4 | 7.9 | 9.0 |
| Vomiting | 3.6 | 3.8 | 3.9 | 7.9* |
| Abdominal | 5.1 | 6.1 | 6.5 | 7.6 |
| Pain | | | | |
| Tremor | 1.5 | 0.9 | 2.5 | 6.7* |
| Anxiety | 1.3 | 2.8 | 4.1 | 5.1 |
| Palpitations | 1.8 | 2.3 | 2.7 | 4.6 |
| Vertigo | 1.8 | 1.6 | 5.0 | 4.5 |
| Fatigue | 3.0 | 2.8 | 2.5 | 4.4 |
| Dysuria | 0.3 | 1.4 | 2.1* | 3.7* |
| Hot flushes | 0 | 1.6 | 3.0 | 3.6 |
| Somnolence | 3.8 | 5.4 | 2.3 | 3.5 |
| Agitation | 3.0 | 1.6 | 3.3 | 2.9 |
| Nervousness | 2.0 | 4.2 | 2.0 | 2.8 |
| Dyspepsia | 4.1 | 3.5 | 2.1 | 2.2 |

*Significantly greater than placebo

It is important to note that in one of the early depression trials, even after one week of milnacipran dose escalation employed to reduce side effects, the most commonly reported reason for discontinuation of treatment because of adverse effects was nausea and vomiting (Leinonen E., 1997, Acta Psychiatr. Scand., 96:497-504). In the recent fibromyalgia clinical trial with the long dose escalation period (four weeks) which was implemented in order to reduce milnacipran side effects and increase patient's tolerance, the most common dose-related side effect reported by patients was nausea (Cypress Bioscience Inc., Cypress Bioscience Inc. Announces Final Results of Milnacipran Phase II Clinical Trial in Fibromyalgia, Media Release, Mar. 21, 2003).

The data presented in Table I demonstrates that the currently available immediate release formulation of milnacipran is not ideal for the treatment of health conditions that require milnacipran doses equal or above 100 mg/day given either as once a day or twice a day due to high incidence of treatment-emergent side effects that leads to poor patient tolerance. Higher doses are required in the treatment of severe depression and other associated disorders. As shown in one of the early antidepressant clinical trials, milnacipran dosage of 200 mg/day was superior to the lower doses (Von Frenckell R et al., 1990, Int. Clin. Psychopharmacology., 5:49-56). Milnacipran dosing regime of 100-250 mg daily was recently reported for the treatment of fibromyalgia (U.S. Pat. No. 6,602,911). It would be very difficult to reach the upper limits of the dose range using the currently available formulation due to the dose related treatment emergent side effects and the need to titrate over a long period to reach the required dose.

Moreover, an immediate release formulation of milnacipran may not be suitable for a once-daily dosing regimen for a treatment of depression due to milnacipran's relatively short, approximately 8 hours, half-life (Ansseau M. et al., 1994, Psychopharmacology, 114:131-137). Milnacipran's half-life could also be responsible for the fact that twice-a-day administration (versus once-a-day) of immediate release formulation in fibromyalgia trial resulted in pain improvement statistically superior to that of placebo treatment (Cypress Bioscience Inc., Cypress Bioscience Inc. Announces Final Results of Milnacipran Phase II Clinical Trial in Fibromyalgia, Media Release, Mar. 21, 2003.

It is therefore an object of the present invention to provide milnacipran formulations which will lower incidence and intensity of side effects, especially for higher dosages, and lower or reduce the frequency of dosing and the need to slowly titrate the drug in order to get to the therapeutic dose levels required for treatment of these disorders.

It is therefore an object of the present invention to provide milnacipran formulations that produce a therapeutic effect over approximately 24 hours when administered to a patient in need, wherein the release rate and dosage are effective to provide relief from at least one disorder selected from the group consisting of depression, fibromyalgia syndrome, chronic fatigue syndrome, pain, attention deficit/hyperactivity disorder, and visceral pain syndromes (VPS), such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache, with diminished incidence and reduced intensity of common milnacipran side effects reported for immediate release formulation.

It is a further object of the present invention to provide formulations that provide alternative pharmacokinetic release profiles that eliminate or diminish unwanted side effects and the current need to slowly increase (titrate) doses in order to achieve the desired therapeutic dose.

It is still another object of the present invention to provide a formulation that provides a unit dose between 25 and 500 mg which provides for flexibility in morning or evening administration.

### SUMMARY OF THE INVENTION

A once-a-day oral milnacipran pulsatile release composition has been developed. This pulsatile composition, when administered orally, releases drug in spaced apart “pulses”. This delivery profile minimizes the exposure of the internal mucosal surfaces to the drug substance and thus reduces milnacipran gastrointestinal side effects such as nausea and vomiting while maintaining therapeutic milnacipran blood plasma levels. Furthermore, this pulsatile composition is ideally suited for the delivery of milnacipran since it has been shown that twice-a-day milnacipran administration results in an enhanced therapeutic response as compared to once-a-day administration. This dosage form provides in vivo drug plasma levels characterized by Cₘₐₓ below 3000 ng/ml, preferably below 2000 ng/ml, and most preferably below 1000 ng/ml. These levels help to avoid stimulation of the cholinergic effects on the CNS. The composition delivers milnacipran over approximately 24 hours, resulting in diminished incidence and decreased intensity of common milnacipran side effects such as nausea, vomiting, sleep disturbance, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

### DETAILED DESCRIPTION OF THE INVENTION

Pulsatile Release Milnacipran Formulations

The composition provides an initial rapid release of a therapeutically effective dose of milnacipran followed by so-called “delayed release” pulses such that a second and optional third delayed dose of the active agent is released from the dosage form. By incorporating both an immediate release dosage unit and one or more delayed release dosage units of the active agent, the dosage form mimics a multiple dosing profile without repeated dosing, i.e., with only a single administration in a day. For example, the dosage form provides a twice daily dosing profile when the dosage form contains both an immediate release dosage unit and a single delayed release dosage unit. Alternatively, the dosage form provides a three times daily dosing profile when the dosage form contains an immediate release dosage unit and two delayed release dosage units.

The formulation provides a pulsatile release dosage form for treating conditions responsive to the administration of milnacipran, wherein the dosage form comprises an immediate release dosage unit, a delayed release dosage unit and an optional second delayed release dosage unit. The immediate release dosage unit comprises a first dose of an active agent that is released substantially immediately following oral administration of the dosage form to a patient. The delayed release dosage unit comprises a second dose of the active agent and a means for delaying release of the second dose until approximately 3 hours to less than 14 hours following oral administration of the dosage form. The second delayed release dosage unit, when present, comprises a third dose of the active agent and a means for delaying release of the third dose until at least 5 hours to approximately 18 hours following oral administration of the dosage form.

Each dosage form contains a therapeutically effective amount of active agent. For dosage forms that mimic the twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 30 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and most preferably approximately 5 hours to 12 hours, following administration.

For dosage forms mimicking the three times daily dosing profile, approximately 25 wt. % to 40 wt. % of the total amount of active agent in the dosage form is released in the initial pulse, and approximately 25 wt. % to 40 wt. % of the total amount of active agent in the dosage form is released in each of the second and third pulses. For dosage forms that mimic the three times daily dosing profile, release of the second pulse preferably takes place approximately 3 hours to 10 hours, and most preferably approximately 4 to 9 hours, following oral administration. Release of the third pulse occurs about 2 hours to about 8 hours following the second pulse, and is typically about 5 hours to approximately 18 hours following oral administration.

In one aspect, a dosage form comprising a closed capsule housing at least two drug-containing dosage units is used. Each dosage unit comprises two or more compressed tablets, or may be comprised of a plurality of beads, granules or particles, providing that each dosage unit has a different drug release profile. The immediate release dosage unit releases drug substantially immediately following oral administration to provide an initial dose. The delayed release dosage unit releases drug approximately 3 hours to 14 hours following oral administration to provide a second dose. Finally, an optional second delayed release dosage unit releases drug about 2 hours to 8 hours following the release of the second dose, and is typically 5 hours to 18 hours following oral administration.

Another dosage form comprises a compressed tablet having a drug-containing immediate release dosage unit, a delayed release dosage unit and an optional second delayed release dosage unit. In this dosage form, the immediate release dosage unit comprises a plurality of beads, granules or particles that release drug substantially immediately following oral administration to provide an initial dose. The delayed release dosage unit comprises a plurality of coated beads or granules, which release drug approximately 3 hours to 14 hours following oral administration to provide a second dose.

An optional second delayed release dosage unit comprises coated beads or granules that release drug about 2 to 8 hours following administration of the initial delayed release dose, typically 5 to 18 hours following oral administration. The beads or granules in the delayed release dosage unit(s) are coated with a bioerodible polymeric material. This coating prevents the drug from being released until the appropriate time, i.e., approximately 3 hours to less than 14 hours following oral administration for the delayed release dosage unit and at least 5 hours to approximately 18 hours following oral administration for the optional second delayed release dosage unit. In this dosage form the components may be admixed in the tablet or may be layered to form a laminated tablet.

Another dosage form is a tablet having a drug-containing immediate release dosage unit, a delayed release dosage unit, and an optional second delayed release dosage unit, wherein the immediate release dosage unit comprises an outer layer that releases the drug substantially immediately following oral administration. The arrangement of the remaining delayed release dosage(s), however, depends upon whether the dosage form is designed to mimic twice daily dosing or three times daily dosing.

In the dosage form mimicking twice daily dosing, the delayed release dosage unit comprises an inner core that is coated with a bioerodible polymeric material. The coating is applied such that release of the drug occurs approximately 3 hours to less than 14 hours following oral administration. In this form, the outer layer completely surrounds the inner core.

In the dosage form mimicking three times a day dosing, the (first) delayed release dose comprises an internal layer that releases drug approximately 3 hours to less than 14 hours following oral administration. This internal layer is surrounded by the outer layer. The second delayed release dosage unit generally comprises an inner core that releases the drug at least 5 hours to approximately 18 hours following oral administration. Thus, the layers of this tablet (starting from the external surface) comprise an outer layer, an internal layer and an inner core. The inner core comprises delayed release beads or granules. Furthermore, the internal layer comprises the drug coated with a bioerodible polymeric material. Alternatively, in this particular dosage form mimicking three times a day dosing, both the delayed release dosage unit and second delayed release dosage units are surrounded by an inner layer. This inner layer is free of active agent. Thus, the layers of this tablet (starting from the external surface) comprise an outer layer, inner layer and an admixture of the delayed release dosage units. The first delayed release pulse occurs once the inner layer is substantially eroded thereby releasing the admixture of the delayed release dosage units. The dose corresponding to the (first) delayed release dosage unit is released immediately since the inner layer has prevented access to this dose for the appropriate time, e.g., from approximately 3 hours to 10 hours. The second delayed release dose, however, is formulated to effectively delay release for at least 5 hours to approximately 18 hours following oral administration.

For formulations mimicking twice daily dosing, it is preferred that the delayed release dose is released approximately 3 hours to less than 14 hours, most preferably approximately 5 hours to 12 hours, following oral administration. For formulations mimicking three times daily dosing, it is preferred that the (first) delayed release dose is released approximately 3 to 10 hours, preferably 4 hours to 9 hours, following oral administration. For dosage forms containing a third dose, the third dose (i.e., the second delayed release dose) is released at least 5 hours to approximately 18 hours following oral administration.

These drug dosage forms are administered orally and can be used for the treatment of depression, for fibromyalgia syndrome, chronic fatigue syndrome, pain, attention deficit/hyperactivity disorder, and visceral pain syndromes (VPS) such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache.

Unless otherwise indicated this formulation and method of use thereof is not limited to specific pharmaceutical carriers or to particular administration regimens, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “an active agent” includes a single active agent as well as combinations of different active agents, reference to “a pharmaceutical carrier” includes a single pharmaceutical carrier as well as combinations of two or more carriers, reference to “a compressed tablet” includes a single as well as a plurality of compressed tablets, reference to “an immediate release dosage form” includes a single immediate release dosage form in addition to a group of two or more immediate release dosage forms, reference to “a coating” as in “a delayed release coating” includes a single coating as well as two or more coatings, and the like.

“Optional” or “optionally” means that the subsequently described circumstance may or may not occur, so that the description includes instances where the circumstance occurs and instances where it does not. For example, when an “optional second delayed release dosage unit” appears in describing the dosage forms, “optional second delayed release dosage unit,” means that the second delayed release dosage unit may or may not be present, and thus, the description includes dosage forms wherein the second delayed release dosage unit is present and dosage forms wherein the second delayed release dosage unit is not present. As used herein, “about” means approximately plus or minus 10%.

The milnacipran composition provides a pulsatile delivery dosage form for administering milnacipran and mimics twice or three times daily dosing of milnacipran. That is, the composition provides an immediate dose followed by one or more pulsatile doses several hours after ingestion of the dosage form.

Optionally, only less than 10% of the first pulse releases substantially immediately after ingestion of the dosage form. More than 90% of the first pulse becomes available after formulation passes through the stomach and enters the small intestines. The composition further provides second and, optionally, third drug doses several hours after ingestion of the dosage form. Since milnacipran is known to cause gastrointestinal disturbances, delay of the first dose should substantially reduce exposure of stomach mucosa to the drug and, hence, diminish milnacipran local side effects such as nausea and vomiting.

The expected therapeutic benefit of these formulations is further supported by the results of a 12-week randomized, double-blind placebo-controlled dose escalation monotherapy trial that evaluated milnacipran in patients with a diagnosis of Fibromyalgia Syndrome (FMS) presented by Cypress Bioscience, Inc. at the 41^{st} Annual Meeting of American College of Neuropsychopharmacology, San Juan, Puerto Rico (Gendreau R. M. et al., Dec. 9, 2002, Poster presentation, Poster #85 “Development of milnacipran, a dual reuptake inhibitor for treatment of chronic pain associated with fibromyalgia”).

In the FMS trial conducted by Cypress Bioscience, all patients were escalated over a 4-week period in weekly steps from 25 mg daily, to 50, 100, and finally 200 mg daily, or until dose-limiting toxicity was evident. The currently available immediate release (IR) milnacipran formulation was used as the only milnacipran dosage form in this study. Patients who successfully reached the 200 mg daily dose were then treated for an additional 8 weeks at that dose. It is important to emphasize that at any given dose level, milnacipran once daily (QD-IR) patients received the full dose of immediate release milnacipran in the morning and received a placebo at night. Milnacipran twice daily (BID-IR) patients received the same total amount in a split dose, given morning and evening.

The primary endpoint used by Cypress Bioscience was defined as the change in pain score from baseline to endpoint based on pain scores collected on the patient electronic diary. Endpoint was defined as week twelve for assessments with a single value (such as clinical measures) or the average of scores at weeks 11 and 12 for diary-based outcomes. It was shown that milnacipran effectively treated pain associated with fibromyalgia syndrome and, additionally, improved mood in depressed patients with FMS. The improvement in pain scores reported by study participants, when 200 mg daily dose was reached, indicates that this substantially higher dose than the one typically used for depression treatment is needed to the alleviation of pain. On a 1-7 scale the global pain scores for all patients who reached endpoint at the time of the analysis, where 1 is very much improved, 4 is unchanged, and 7 is very much worse, the mean value for milnacipran patients was 2.3, while the mean value for placebo patients was 4.3 (the difference between the milnacipran groups and placebo is statistically significant at p=0.0001). Importantly, within the milnacipran groups, twice daily dosing was significantly more effective than once daily dosing in pain reduction. Twice daily dosing regimen in addition to being more therapeutically effective, also demonstrated fewer dose-related adverse events and resulted in a lower rate of dose intolerance than once daily regimen (19% of participants in QD-IR group failed the dose escalation vs. only 6% in BID-IR group). Note that no dose escalation failures were recorded in the placebo group.

These clinical differences between QD-IR and BID-IR are most likely due to the distinct differences in the drug plasma levels (especially Cₘₐₓ) that these two dosing regiments support. The BID-IR dosing regimen supports drug plasma levels characterized by lower Cₘₐₓ and lower drug plasma fluctuations over 24 hour time period than that of QD-IR. When a daily dose is administered QD-IR, the Cₘₐₓ is approximately twice higher than that of BID-IR dosing regimen. Higher Cₘₐₓ causes an increase in the severity of the adverse side effects (that also might interfere with the objective pain level self-assessment by the patient) and leads to a lower drug tolerance and patient compliance. Therefore, the observed superior milnacipran performance when drug was administered BID-IR is thought to be due to more “sustained” drug plasma levels over a 24 hour period.

Based on the clinical trial data obtained and presented by Cypress Bioscience, sleep quality improves, albeit marginally, when milnacipran was administered BID-IR. This could be interpreted as another indication that the formulation that provides more “sustained” drug plasma levels over a 24 hour period should demonstrate superior performance when compared to standard immediate release formulation and, importantly, cause less insomnia.

Definitions

Delayed release dosage form: A delayed release dosage form is one that releases a drug (or drugs) at a time other than promptly after administration.

Pulsatile release dosage form: A pulsatile release dosage form is one that mimics a multiple dosing profile without repeated dosing and allows at least a twofold reduction in dosing frequency as compared to that drug presented as a conventional dosage form (e.g. as a solution or prompt drug-releasing, conventional solid dosage form). A pulsatile release profile is characterized by a time period of no release (lag time) followed by rapid drug release.

Milnacipran

Milnacipran and methods for its synthesis are described in U.S. Pat. No. 4,478,836. Milnacipran (midalcipran, midacipran, F 2207) inhibits the uptake of both, norepinephrine (NE) and serotonin (5-HT), with an NE to 5-HT ratio of 2:1 (Moret et al., 1985, Neuropharmacology, 24:1211-1219; Palmier et al., 1989, Eur. J. Clin. Pharmacol., 37:235-238) but does not affect the uptake of dopamine. Milnacipran has no affinity for alpha or beta adrenergic, muscarinic, histaminergic, and dopaminergic receptors. This suggests that milnacipran has a low potential to produce anticholinergic, sedative, and stimulant effects. Milnacipran does not affect the number of beta adrenoceptors in rat cortex after chronic administration (Briley M. et al., Int. Clin. Psychopharmac., 1996, 11:10-14). Additional information regarding milnacipran may be found in the Merck Index, 12^{th} Edition, at entry 6281.

As used herein “milnacipran” also encompasses pharmaceutically acceptable, pharmacologically active derivatives of milnacipran including both individual enantiomers of milnacipran (dextrogyral and levrogyral enantiomers) and their pharmaceutically acceptable salts, mixtures of milnacipran enantiomers and their pharmaceutically acceptable salts, and active metabolites of milnacipran and their pharmaceutically acceptable salts, unless otherwise noted. It is understood that in some cases dosages of enantiomers, derivatives, and metabolites may need to be adjusted based on relative activity of the racemic mixture of milnacipran.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization of diastereomeric salts/derivatives or preparative chiral chromatography.

Combinations with Other Active Compounds

The milnacipran can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

Specific examples of compounds that can be adjunctively administered with milnacipran include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil, molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

By adjunctive administration is meant simultaneous administration of the compounds, in the same dosage form, simultaneous administration in separate dosage forms, and separate administration of the compounds.

Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et.al., (Media, Pa.: Williams and Wilkins, 1995) which provides information on carriers, materials, equipment and process for preparing tablets and capsules and delayed and/or pulsatile release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides. Other polymers that can be used include biodegradable polymers such as polyhydroxyacids like poly(lactic acid-glycolic acid), and other approved hydrolytically or enzymatically degradable polymers.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, for example, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose,including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The amount of active agent released in each dose will be a therapeutically effective amount. In the case of milnacipran, the total amount in the dosage form is in the range of approximately 25 to 500 mg.

The pharmaceutical dosage forms provide pulsatile delivery of milnacipran. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In one embodiment, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule may comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

For dosage forms mimicking a twice a day dosing profile, a first group beads, granules or particles releases drug substantially immediately following ingestion of the dosage form, while a second group of beads or granules preferably releases drug approximately 3 hours to less than 14 hours following ingestion of the dosage form. For dosage forms mimicking a three times daily dosing profile, a first group of beads, granules or particles releases drug substantially immediately following ingestion of the dosage form, a second group of beads or granules preferably releases drug approximately 3 hours to 10 hours following ingestion of the dosage form, and a third group of beads, granules or particles releases drug at least 5 hours to approximately 18 hours following ingestion of the dosage form. The above-mentioned tablets, beads, granules or particles of different drug release profiles (e.g., immediate and delayed release profiles) may be mixed and included in a capsule, tablet or matrix to provide a pulsatile dosage form having the desired release profile.

In another embodiment, the individual dosage units are compacted in a single tablet, and may represent integral but discrete segments thereof (e.g., layers), or may be present as a simple admixture. For example, drug-containing beads, granules or particles with different drug release profiles (e.g., immediate and delayed release profiles) can be compressed together into a single tablet using conventional tableting means.

In a further alternative embodiment, a dosage form is provided that comprises an inner drug-containing core and at least one drug-containing layer surrounding the inner core. An outer layer of this dosage form contains an initial, immediate release dose of the drug. For dosage forms mimicking twice daily dosing, the dosage form has an outer layer that releases drug substantially immediately following oral administration and an inner core having a polymeric-coating that preferably releases the active agent approximately 3 hours to less than 14 hours following ingestion of the dosage unit. For dosage forms mimicking three times daily dosing, the dosage form has an outer layer that releases drug substantially immediately following oral administration, an inner core that preferably releases drug at least 5 hours to 18 hours following oral administration and a layer interposed between the inner core and outer layer that preferably releases drug approximately 3 hours to 10 hours following ingestion of the dosage form. The inner core of the dosage form mimicking three times daily a dosing may be formulated as compressed delayed release beads or granules.

Alternatively, for dosage forms mimicking three times daily dosing, the dosage form has an outer layer and an inner layer free of drug. The outer layer releases drug substantially immediately following oral administration, and completely surrounds the inner layer. The inner layer surrounds both the second and third doses and preferably prevents release of these doses for approximately 3 hours to 10 hours following oral administration. Once released, the second dose is immediately available while the third dose is formulated as delayed release beads or granules such that release of the third dose is effected approximately 2 hours to 8 thereafter effectively resulting in release of the third dose at least 5 hours to approximately 18 hours following ingestion of the dosage form. The second and third doses may be formulated by admixing immediate release and delayed release beads, granules or particles and compressing the admixture to form a second and third dose-containing core followed by polymeric coating to achieve the desired three times daily dosing profile.

In still another embodiment, a dosage form comprises a coated core-type delivery system wherein the outer layer is comprised of an immediate release dosage unit, such that active agent therein is immediately release following oral administration, an intermediate layer thereunder surrounds a core, and the core is comprised of immediate release beads or granules and delayed release beads or granules, such that the second dose is provided by the immediate release beads or granules and the third dose is provided by the delayed release beads or granules.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily a incorporating a polymeric material; increasing drug particle size; placing the drug within a matrix; and forming complexes of the drug with a suitable complexing agent.

The delayed release dosage units in any of the above embodiments can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials are comprised of bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit.RTM. (Rohm Pharma; Westerstadt, Germany), including Eudragit.RTM. L30D-55 and L100-55 (soluble at pH 5.5 and above), Eudragit.RTM. L-100 (soluble at pH 6.0 and above), Eudragit.RTM. S (soluble at pH 7.0 and above, as a result of a higher degree of esterification), and Eudragits.RTM. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers are, but not limited to, polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage, reference may be made to Pharmaceutical Dosage Forms: Tablets, eds. Lieberman et al. (New York: Marcel Dekker, Inc., 1989), and to Ansel et al., Pharmaceutical Dosage Forms and Drug Delivery Systems, 6.sup.th Ed. (Media, P A: Williams & Wilkins, 1995).

Alternatively, a delayed release tablet may be formulated by dispersing the drug within a matrix of a suitable material such as a hydrophilic polymer or a fatty compound. The hydrophilic polymers may be comprised of polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

The immediate release dosage unit of the dosage form—i.e., a tablet, a plurality of drug-containing beads, granules or particles, or an outer layer of a coated core dosage form—contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads). A preferred method for preparing immediate release tablets (e.g., as incorporated into a capsule) is by compressing a drug-containing blend, e.g., blend of granules, prepared using a direct blend, wet-granulation or dry-granulation process. Immediate release tablets may also be molded rather than compressed, starting with a moist material containing a suitable water-soluble lubricant. However, preferred tablets herein are manufactured using compression rather than molding. A preferred method for forming immediate release drug-containing blend is to mix drug particles directly with one or more excipients such as diluents (or fillers), binders, disintegrants, lubricants, glidants, colorants or the like. As an alternative to direct blending, a drug-containing blend may be prepared by using a wet-granulation or dry-granulation processes. Beads containing the active agent may also be prepared by any one of a number of conventional techniques, typically starting from a fluid dispersion. For example, a typical method for preparing drug-containing beads involves blending the active agent with conventional pharmaceutical excipients such as microcrystalline cellulose, starch, polyvinylpyrrolidone, methylcellulose, talc, metallic stearates, silicone dioxide, or the like. The admixture is used to coat a bead core such as a sugar sphere (or so-called “non-pareil”) having a size of approximately 20 to 60 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Optional pharmaceutically acceptable excipients present in the drug-containing tablets, beads, granules or particles include, but are not limited to, diluents, binders, lubricants, disintegrants, colorants, stabilizers, surfactants and the like. Diluents, also termed “fillers,” are typically necessary to increase the bulk of a solid dosage form so that a practical size is provided for compression of tablets or formation of beads and granules. Suitable diluents include, for example, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar. Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Lubricants are used to facilitate tablet manufacture; examples of suitable lubricants include, for example, magnesium stearate, calcium stearate, stearic acid, glycerol behenate, and polyethylene glycol, talc, and mineral oil. Disintegrants are used to facilitate dosage form disintegration or “breakup” after administration, and are generally starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp). Stabilizers are used to inhibit or retard drug decomposition reactions which include, by way or example, oxidative reactions. Surfactants may be anionic, cationic, amphoteric or nonionic surface active agents. Suitable anionic surfactants include, but not limited to those containing carboxylate, sulfonate and sulfate ions. Examples for anionic surfactants are sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but not limited quaternary ammonium compounds such as benzalkonium chloride, benzethonium chloride, cetrimonium bromide, stearyl dimethylbenzyl ammonium chloride, polyoxyethylene (15) and coconut amine. Examples for nonionic surfactants are, but not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene (8) monolaurate, polysorbates, ii polyoxyethylene (9) octylphenylether, PEG-1000 cetyl ether, polyoxyethylene (3) tridecyl ether, polypropylene glycol (18) butyl ether, Poloxamer 401, stearoyl monoisopropanolamide, and polyoxyethylene (5) hydrogenated tallow amide. Examples for amphoteric surfactants are, but not limited to, sodium N-dodecyl-.beta.-alanine, sodium N-lauryl-.beta.-iminodipropionate, myristoamphoacetate, lauryl betaine and lauryl sulfobetaine. If desired, the tablets, beads granules or particles may also contain minor amount of nontoxic auxiliary substances such as wetting or emulsifying agents, pH buffering agents, preservatives, and the like.

The amount of active agent released in each dose will be a therapeutically effective amount. In the case of milnacipran, the total amount in the dosage form is in the range of approximately 25 to 500 mg. Typically, the total amount of active agent in a dosage form is divided evenly between each pulse contained in the dosage form. For dosage forms that mimic a twice a day profile, the active agent in immediate release form generally represents about 30 wt. % to 70 wt. %, preferably 40 wt. % to 60 wt. %, of the total active agent in one dosage form, while, correspondingly, the active agent in the delayed release form generally represents about 70 wt. % to 30 wt. %, preferably 60 wt. % to 40 wt. %, of the total active agent in one dosage form. Similarly, for dosage forms that mimic three times daily dosing profile, the active agent in the immediate release unit(s) and in each of the two delayed release units represents about 20 wt. % to 50 wt. %, preferably 25 wt. % to 40 wt. %, of the total active agent in one dosage form.

All publications mentioned herein are incorporated by reference in their entireties.

Kit Containing Pulsatile Release Formulations

A kit is provided wherein the once a day pulsatile release dosage form is packaged to provide a method to conveniently begin dose titration at lower doses, for example, beginning at 25 mg, gradually increasing to 50 mg, 75 mg, 100 mg, 200 mg, 400 mg, 500 mg, over a period ranging from three days up to 16 weeks. The kit wherein the packaging material may be a box, bottle, blister package, tray, or card. The kit will include a package insert instructing the patient to take a specific dose at a specific time, for example, a first dose on day one, a second higher dose on day two, a third higher dose on day three, and so on, until a maintenance dose is reached.

Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The pulsatile release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

Administration of Milnacipran Formulations

The formulation can be administered to any patient in need thereof. Although preferred patients are human, typically any mammal including domestic animals such as dogs, cats and horses, may also be treated.

The amount of the active ingredients to be administered is chosen based on the amount which provides the desired dose to the patient in need of such treatment to alleviate symptoms or treat a condition.

Milnacipran has been used as an antidepressant in approximately 400,000 patients, and is known to be non-toxic in humans. Pharmacokinetic studies have shown that oral doses of milnacipran are rapidly absorbed and extensively distributed in the body within 1-2 hours. Maximum plasma levels are quickly reached, with a half-life in humans of approximately 8 hours. Metabolism in the liver leads to the formation of ten chemically identified metabolites, although these metabolites represent only about 10% of the concentration of the parent drug. In humans, 90% of the parent drug is eliminated unchanged via the kidneys. This pharmacokinetic profile gives milnacipran certain pharmacokinetic advantages, such as low inter-individual variation in plasma levels, low potential for drug interactions, and limited impact on hepatic cytochrome P-450 systems. These pharmacokinetic properties differentiate milnacipran from most other antidepressant drugs and contribute to the good safety profile of milnacipran (Puozzo C. et al., 1996, Int. Clin. Psychopharmacol., 11:15-27; Caccia S., 1998, Clin. Pharmacokinet., 34:281-302; Puozzo C. et al., 1998, Eur. J. Drug Metab. Pharmacokinet., 23:280-286).

Milnacipran can be administered for the treatment of depression, for fibromyalgia syndrome, chronic fatigue syndrome, pain, attention deficit/hyperactivity disorder, and visceral pain syndromes (VPS) such as irritable bowel syndrome (IBS), noncardiac chest pain (NCCP), functional dyspepsia, interstitial cystitis, essential vulvodynia, urethral syndrome, orchialgia, and affective disorders, including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, migraine headache, and tension headache.

Adverse reactions to the oral administration of milnacipran typically include at least one of the following: nausea, vomiting, headache, dyspepsia, abdominal pain, insomnia, tremulousness, anxiety, panic attack, palpitations, urinary retention, orthostatic hypotension, diaphoresis, chest pain, rash, weight gain, back pain, constipation, vertigo, increased sweating, agitation, hot flushes, tremors, fatigue, somnolence, dysoria, nervousness, dry mouth, and irritability.

The vomiting reflex is triggered by stimulation of chemoreceptors in the upper GI tract and mechanoreceptors in the wall of the GI tract which are activated by both contraction and distension of the gut wall as well as by physical damage. A coordinating center in the central nervous system controls the emetic response. The center is located in the parvicellular reticular formation in the lateral medullary region of the brain. Afferent nerves to the vomiting center arise from the abdominal splanchic and vagal nerves, vestibule-labyrinthine receptors, the cerebral cortex and the cehmoreceptors trigger zone (CTZ). The CTZ lies adjacent in the area postrema and contains chemoreceptors that sample both blood and cerebro spinal fluid. Direct links exist between the emetic center and the CTZ. The CTZ is exposed to emetic stimuli of endogenous origin and to stimuli of exogenous origin such as drugs. The efferent branches of the cranial nerves V, VII, and IX, as well as the vagus nerve and sympathetic trunk produce the complex coordinated set of muscular contractions, cardiovascular responses and reverse peristalsis that characterizes vomiting. The area postrema is rich in dopamine receptors as well as 5-hydroxytryptamine (5HT) receptors.

When administered orally, the formulation provides an immediate dose followed by one or more pulsatile doses several hours after ingestion of the dosage form. The pharmaceutical composition of milnacipran provides the in vivo drug plasma levels characterized by the first initial peak plasma concentration (Cₘₐₓ) below 3000 ng/ml, preferably below 2000 ng/ml, and most preferably below 1000 ng/ml that occurs in about 0.05 to about 3 hours after oral administration (first Tₘₐₓ). The second in vivo plasma concentration peak occurs in about 3 to about 14 hours after oral administration (second Tₘₐₓ) and is characterized by Cₘₐₓ below 3000 ng/ml, preferably below 2000 ng/ml, and most preferably below 1000 ng/ml. The third, optional, in vivo plasma concentration peak occurs in about 5 to about 18 hours after oral administration (third Tₘₐₓ) and is characterized by Cₘₐₓ below 3000 ng/ml, preferably below 2000 ng/ml, and most preferably below 1000 ng/ml.

When enteric coating is added to the formulation described above, the formulation after oral administration first passes through the stomach releasing less than approximately 10% of the first “pulse” milnacipran dose and then enters the intestines where the remaining portion of the first “pulse” is released. The release profile is characterized by a 0.05-4 hours lag time period during which less than approximately 10% of the first “pulse” milnacipran dose is released followed by a complete release of the first “pulse”. The use of enteric coating minimizes direct milnacipran interaction with the stomach mucosa and, thus, further diminishes incidence and reduces intensity of common milnacipran side effects. An enteric coated formulation demonstrates similar in vivo plasma Cₘₐₓ levels to the uncoated one, but Tₘₐₓ for the first pulse increases by 0.05-4 hours. Tₘₐₓ for the second and the third (optional) pulses may be kept the same as for an uncoated formulation by adjusting the formulation ingredients to meet the desired release profile.

This dosage form offers many advantages, when compared to immediate release delivery systems, such as: minimization of peak-trough-fluctuations, avoidance of undesirable side effects and/or lowering their intensity/severity, reduced frequency of administration and improved patient compliance.

This formulation is designed to be administered once-a-day to a patient in need thereof, so that milnacipran is delivered over approximately 24 hours, with diminished incidence and decreased intensity of one or more common milnacipran side effects such as nausea, vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

### EXEMPLIFICATION

The present invention will be further understood by reference to the following non-limiting examples.

### Example 1

Preparation of an Immediate Release Portion of Pulsatile Release Milnacipran Formulation

Ingredients, manufacturing process, and tablet parameters for the immediate release portion of the pulsatile release milnacipran pharmaceutical composition (Lot #1).

| Ingredient | Quantity per tablet, mg |
|---|---|
| Milnacipran HCl | 50.00 |
| Microcrystalline Cellulose (Avicel PH 101) | 10.00 |
| Pre-gelatinized Starch (Starch 1500) | 10.00 |
| Purified Water | QS |
| Magnesium Stearate | 0.35 |

The formulations were prepared using aqueous media for wet granulation step. To prepare an immediate release tablet, weighed quantities of milnacipran hydrochloride, microcrystalline cellulose, and pre-gelatinized starch were mixed. Purified water was added slowly, while mixing. The wet mass was forced through a #12 mesh screen. Obtained wet granules were dried on a tray dryer at 50° C. and then passed through a #30 mesh screen. Finally, dried granules were lubricated by mixing with magnesium stearate and the blend was then compressed on a 16 station single rotary compression machine.

| Tablet parameter | Lot# 1 |
|---|---|
| Weight (mg) | 69-72 |
| Thickness (Inches) | 0.123-0.127 |
| Diameter (Inches) | 0.2187 |
| Hardness (kP) | 4.5-5.5 |
| Friability (%) | 0.15 |
| Disintegration time | 5 minutes |
| in water | |

### Example 2

Preparation of an Alternative Immediate Release Portion of Pulsatile Release Milnacipran Formulation

Ingredients, manufacturing process, and tablet parameters for an alternative immediate release portion of the pulsatile release milnacipran pharmaceutical composition (Lot #2).

| Ingredient | Quantity per tablet, mg |
|---|---|
| Milnacipran HCl | 50.00 |
| Microcrystalline Cellulose (Avicel PH 101) | 10.00 |
| Pre-gelatinized Starch (Starch 1500) | 10.00 |
| Purified Water | QS |
| Magnesium Stearate | 0.35 |

The formulations were prepared as described above.

| Tablet parameter | Lot# 2 |
|---|---|
| Weight (mg) | 72-74 |
| Thickness (Inches) | 0.168-170 |
| Diameter (Inches) | 0.1875 |
| Hardness (kP) | 5-6 |
| Friability (%) | 0.08% |
| Disintegration time | 5.5 minutes |
| in water | |

### Example 3

Preparation of an Enteric Coated Portion of Pulsatile Release Milnacipran Formulation

Ingredients, manufacturing process, and in vitro dissolution data for the enteric coated portion of the pulsatile release milnacipran pharmaceutical composition.

Lot #1 immediate release tablets were used for preparation of enteric coated dosage form. The manufacturing procedure consisted of spraying an aqueous enteric coating suspension onto the immediate release tablets fluidized in the GPCG-1 (Glatt Air Techniques, Inc.). 20% coat weight gain was achieved for Lot #3. The process parameters were adjusted to accomplish good quality coating. The ingredients of aqueous enteric coating suspension are given below.

| | | Quantity per |
|---|---|---|
| Ingredient | Manufacturer | batch, g |
| Acryl-Eze White | Colorcon | 98.00 |
| Dow Corning ® 7-9245 30% | Dow Corning | 0.490 |
| Simethicone Emulsion USP | | |
| FD&C Blue # 1 Lake Concentrate | Warner | 0.10 |
| | Jenkinson | |
| D & C Red # 33 Aluminum Lake | Warner | 0.10 |
| | Jenkinson | |
| Purified Water USP | | 392.00 |

In vitro dissolution data for Lot #3 enteric coated tablets is given below. In vitro drug release studies were conducted using USP dissolution apparatus II (paddles) at 50 rpm. Experiments were conducted in dissolution media (37.0±0.5° C.), first for 2 hours in 0.1 N hydrochloric acid, followed by 1.5 hours in pH 6.8 phosphate buffer (see USP 26<724> Method B for Delayed Release Articles). Sample aliquots were withdrawn after 2 hours in 0.1 N hydrochloric acid, and after 15, 30, 45 and 90 minutes in pH 6.8 buffer and analyzed using the HPLC method.

| | |
|---|---|
| | Lot# 3 |
| Cumulative | Milnacipran released, % of the total dose |

| incubation time, min | Mean values (n = 6) | Standard deviation |
|---|---|---|
| 0.1 N HCl | | |
| 120 | NS | NA |
| pH 6.8 buffer | | |
| 135 | 6.0 | 5.6 |
| 150 | 80.0 | 5.5 |
| 165 | 97.8 | 0.8 |
| 210 | 98.5 | 0.8 |

NS - less than 1% was detected
NA - Not applicable

### Example 4

Preparation of a Delayed Release Portion of Pulsatile Release Milnacipran Formulation

Ingredients, manufacturing process, and in vitro dissolution data for the delayed release portion of the pulsatile release milnacipran pharmaceutical composition.

Lot #2 immediate release tablets were used for preparation of delayed release dosage form. The manufacturing procedure consisted of spraying an aqueous coating suspension onto the immediate release tablets fluidized in the GPCG-1 (Glatt Air Techniques, Inc.). 30% coat weight gain was achieved for Lot #4. The process parameters were adjusted to accomplish good quality coating. After coating process was completed, tablets were further dried in the GPCG-1 for 30 minutes at 40° C. followed by 60 hours of “curing” at 30° C. in the oven drier. The “curing” time could be shortened by increasing the drying temperature, for example, only 6 hours of drying is needed at 50° C. The ingredients of aqueous enteric coating suspension are given below.

| | | Quantity |
|---|---|---|
| Ingredient | Manufacturer | per batch, g |
| Eudragit S 100 Powder | Rohm Pharma | 268.8 |
| | Polymers | |
| 1 N Ammonia solution in water (1.7%) | Spectrum | 136.8 |
| Triethyl Citrate FCC | Spectrum | 188.2 |
| Talc USP | Spectrum | 26.8 |
| Purified Water USP | | 1584.0 |

The samples were subjected to the in vitro dissolution tests that mimic the in vivo conditions to which final dosage form is exposed when administered orally, i.e. approximately 2 hours in the stomach at acidic pH followed by several hours in the intestines at neutral pH (Multiparticulate Oral Drug Delivery, 1994, Ghebre-Sellassie I., Ed., Marcel Dekker, Inc.; Wilding I. R., 2001, Adv. Drug Deliv. Rev., 46:103-124). In vitro drug release studies were conducted using USP dissolution apparatus II (paddles) at 50 rpm. Experiments were conducted in dissolution media (37.0±0.5° C.) first for 2 hours in 0.1 N hydrochloric acid followed by 0.5 hours in pH 6.0 phosphate buffer, 4 hours in pH 6.5 phosphate buffer and, finally, 4 hours in pH 7.2 phosphate buffer. Samples were withdrawn and analyzed using validated UV method (in a separate experiment it was shown that the UV method gives substantially the same results as the HPLC method).

| | |
|---|---|
| Cumulative | Lot# 4 |
| incubation | Milnacipran released, % of the total dose |

| time, hours | Mean values (n = 3) | Standard deviation |
|---|---|---|
| 0.1 N HCl | | |
| 2 | NS | NA |
| pH 6.0 buffer | | |
| 2.5 | NS | NA |
| pH 6.5 buffer | | |
| 6.5 | NS | NA |
| pH 7.2 buffer | | |
| 7 | NS | NA |
| 7.5 | NS | NA |
| 8 | 5.1 | 1.3 |
| 8.5 | 26.7 | 3.2 |
| 9 | 91.4 | 4.7 |
| 9.5 | 104.9 | 1.1 |
| 10.5 | 104.2 | 0.5 |

NS - less than 1% was detected
NA - Not applicable

### Example 5

Preparation of a Pulsatile Release Milnacipran Formulation

The final dosage form for pulsatile delivery of milnacipran is prepared by combining in the desired proportion an immediate release portion of the Examples 1 or 2 with a delayed release portion of the Example 4. Different size capsules could be prepared depending on the required daily dose.

### Example 6

Preparation of an Alternative Pulsatile Release Milnacipran Formulation

The final dosage form for pulsatile delivery of milnacipran is prepared by combining in the desired proportion an enteric coated portion of the Example 3 with a delayed release portion of the Example 4. Different size capsules could be prepared depending on the required daily dose.

We claim:

1. A milnacipran formulation that provides pulsatile release of milnacipran to produce a therapeutic effect over approximately 24 hours when administered to a patient in need, with diminished incidence and reduced intensity relative to one or more immediate release milnacipran side effects.

2. The milnacipran formulation according to claim 1, wherein the side effect is nausea.

3. The malnacipran formulation according to claim 1, wherein the side effects are selected from the group consisting of vomiting, headache, tremulousness, anxiety, panic attacks, palpitations, urinary retention, orthostatic hypotension, diaphoresis, chest pain, rash, weight gain, back pain, constipation, vertigo, increased sweating, agitation, hot flushes, tremors, fatigue, somnolence, dyspepsia, dysoria, nervousness, dry mouth, abdominal pain, irritability, and insomnia.

4. The milnacipran formulation according to claim 1 comprising:
(a) an immediate release dosage unit comprising a first dose of the active agent that is released substantially immediately following oral administration of the dosage form to a patient resulting in the first plasma level peak at approximately 0.05 hours to less than 3 hours following oral administration;
(b) a delayed release dosage unit comprising a second dose of the active agent and a means for delaying release of the second dose resulting in the second plasma level peak at approximately 3 hours to less than 14 hours following oral administration of the dosage form; and optionally
(c) a second delayed release dosage unit comprising a third dose of the active agent and a means for delaying release of the third dose resulting in the third plasma level peak at approximately 5 hours to less than 18 hours following oral administration of the dosage form.

5. The milnacipran formulation according to claim 4 wherein an enteric coating is added to the formulation and the release profile is further characterized by a 0.05-4 hours lag time period during which less than approximately 10% of the first “pulse” milnacipran dose is released followed by a complete release of the first “pulse”.

6. The milnacipran formulation according to claim 1 providing milnacipran blood plasma levels that are characterized by Cₘₐₓ below approximately 3000 ng/ml.

7. The milnacipran formulation according to claim 6 providing milnacipran blood plasma levels that are characterized by Cₘₐₓ below approximately 2000 ng/ml.

8. The milnacipran formulation according to claim 6 providing milnacipran blood plasma levels that are characterized by Cₘₐₓ below approximately 1000 ng/ml.

9. The milnacipran formulation according to claim 1 further comprising at least one other active compound selected from the group consisting of analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

10. The milnacipran formulation according to claim 9 comprising one or more compounds selected from the group consisting of aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil, molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

11. The milnacipran formulation according to claim 1, wherein the milnacipran is in the form of a therapeutically equivalent dose of dextrogyral or levrogyral enantiomers of the milnacipran or pharmaceutically acceptable salts thereof.

12. The milnacipran formulation according to claim 1, wherein the milnacipran is in the form of a therapeutically equivalent dose of a mixture of milnacipran enantiomers or pharmaceutically acceptable salts thereof.

13. The milnacipran formulation according to claim 1, wherein the milnacipran is in the form of a therapeutically equivalent dose of the active metabolite of milnacipran or pharmaceutically acceptable salts thereof.

14. The milnacipran formulation according to claim 1, wherein the milnacipran is in the form of a therapeutically equivalent dose of para-hydroxy-milnacipran (F2782) or pharmaceutically acceptable salts thereof.

15. The milnacipran formulation according to claim 1 comprising an enteric coating.

16. The milnacipran formulation according to claim 1, wherein the administrable milnacipran unit dose is from 25 to 500 mg.

17. The milnacipran formulation according to claim 1, wherein the administrable milnacipran unit dose is from 200 to 500 mg.

18. The milnacipran formulation according to claim 9 comprising 25 to 500 mg milnacipran and 100 to 600 mg modafinil.

19. The milnacipran formulation according to claim 1 comprising a mixture of beads or particles releasing drug at different times.

20. A kit comprising the milnacipran formulation of claim 1.

21. The kit of claim 20 comprising different dosage units of milnacipran to allow for dosage escalation.

22. The kit of claim 20 comprising instruction on taking the formulation once daily before bedtime.

23. A method of making a milnacipran formulation comprising providing the formulation of claim 1.

24. A method for delivering a therapeutic dose of milnacipran as a starting dose to a patient in need thereof, with diminished incidence or reduced intensity of common milnacipran side effects, comprising administering to the patient in need thereof the milnacipran formulation of claim 1.
